(19) United States
(12) Patent Application Publication
(10) Pub. No.: US 2011/0282325 A1
(43) Pub. Date: Nov. 17, 2011

(54) DEHYDRATED PLASMA KIT

(75) Inventor: Kenton W. Gregory, Portland, OR (US)

(73) Assignee: Ore-gon Biomedical Engineering Institute, Portland, OR (US)

(21) Appl. No.: 13/145,865

(22) PCT Filed: Jan. 26, 2010
(86) PCT No.: PCT/US10/22080

§ 371 Date: Jul. 22, 2011

Related U.S. Application Data
(60) Provisional application No. 61/147,167, filed on Jan. 26, 2009.

(51) Int. Cl. A61M 5/31; A61J 1/06

(52) U.S. Cl. 604/518; 604/404; 604/257

(57) ABSTRACT

The present invention relates to a portable kit and methods for administering a therapeutic dose of autologous plasma to an injured person.

### TECHNICAL FIELD

Embodiments of the present invention relates to the field of trauma care.

### BACKGROUND

Plasma, a clear yellow liquid that contains the clotting proteins needed to stop bleeding in the injured, is a component of blood. In modern hospitals, it is stored frozen at a temperature no higher than −18 degrees Celsius for up to one year before thawing and use. Plasma is generally available in emergency rooms. However, for persons injured in remote areas, plasma is not available until they get to a hospital potentially hours after the injury.

In spite of extensive precautions, recent concerns with the possibility of disease transmission from the use of blood products obtained from sources foreign to the patient have severely limited the use of nonautologous plasma. Accordingly, there exists a need for obtaining, storing and administering autologous plasma to persons injured in remote areas.

### BRIEF DESCRIPTION OF THE DRAWINGS

Embodiments of the present invention will be readily understood by the following detailed description in conjunction with the accompanying figures.

FIG. 1 is a simplified illustration of a portable kit for administering a therapeutic dose of autologous plasma.

FIG. 2 is a simplified illustration of a system for separating whole plasma from a sample of blood.

### DETAILED DESCRIPTION OF EMBODIMENTS OF THE INVENTION

In the following detailed description, reference is made to embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete steps in turn, in a manner that may be helpful in understanding embodiments of the present invention; however, the order of description should not be construed to imply that these operations are order dependent.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments of the invention, methods and kits for administering a therapeutic dose of autologous plasma to an injured person are provided. Although certain embodiments have been described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

Various embodiments of the present invention relate to a portable kit for storing and administering a therapeutic dose of autologous plasma to an injured person. In the case of an injured soldier, for example, the portable kit may be constructed to be lightweight and capable of fitting into a soldier's combat uniform pocket and/or modular lightweight load carrying equipment (i.e., MOLLE). In this way, each soldier may carry a therapeutic quantity of his or her own plasma for use in the unfortunate event of a battlefield injury.

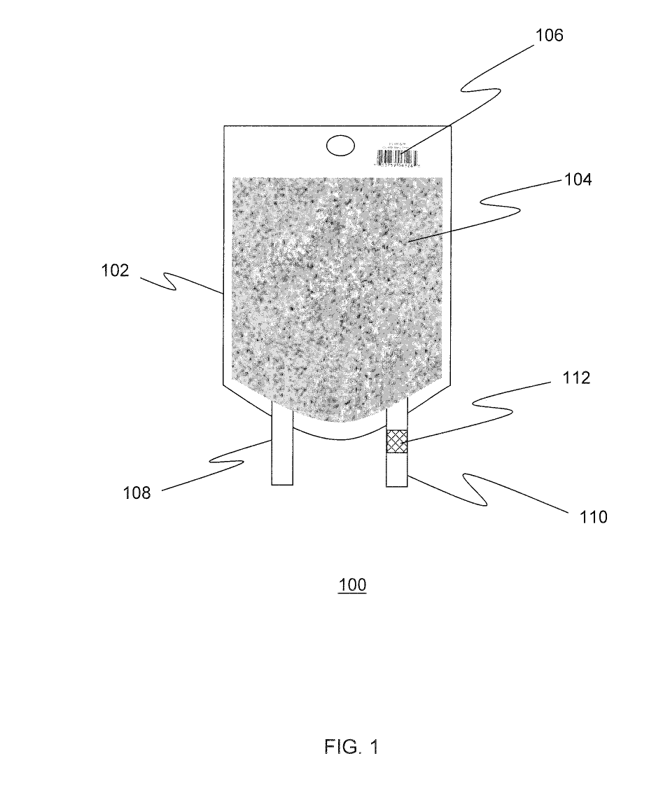

Referring to FIG. 1, wherein a simplified diagram illustrating an embodiment of the present invention is shown, a plasma kit 100 may include a plasma container 102 filled with a predetermined quantity of dehydrated plasma 104. In various embodiments, dehydrated plasma 104 is sterile. In still further embodiments, dehydrated plasma 104 is freeze-dried plasma.

In various embodiments, dehydrated plasma 104 may be capable of producing a therapeutic dose of plasma when rehydrated. For such embodiments, the predetermined quantity of dehydrated plasma 104 may be in the range of 30 g to 140 g.

In accordance with various embodiments of the invention, the injured person's own blood may be the source of dehydrated plasma 104 (i.e., dehydrated plasma 104 is autologous plasma). To identify the injured person as the sole source of the dehydrated plasma, plasma kit 100 may include a tag 106 having identifying information. In various aspects of the present invention, the identifying information may be in the form of, but not limited to, text, magnetic stripe, Radio-frequency-identification (RFID) and/or barcode or other optical code.

In various embodiments of the present invention, container 102 may include a fluid inlet 108 for filling plasma container 102 with fluid to rehydrate dehydrated plasma 104. In further embodiments, plasma container 102 may include a fluid outlet 110 for administering the rehydrated autologous plasma to the injured donor. For such embodiments, fluid outlet 110 may include a filter 112 for removing non-rehydrated plasma particulates.

In various embodiments, plasma container 102 may be an intravenous fluid bag. In embodiments, the volume of plasma container 102 may be in the range of 400 ml to 2000 ml.

In further embodiments, plasma container 102 may be stored in an outer container. In such embodiments, the outer container may be a cylinder, bag or other suitable container. The outer container may serve to protect plasma container 102 and/or facilitate carrying or attaching plasma kit 100 to clothing or load carrying equipment. In exemplary embodiments, tag 106 may be coupled to plasma container 102 and/or the outer container.

Various embodiments of the present invention include methods for administering a therapeutic dose of autologous plasma to an injured person employing the portable kit of FIG. 1. According to such embodiments, a sample of blood may be obtained from a single donor. Then, a whole plasma fraction may be separated from the sample of blood. Next, the whole plasma may be dried to form dehydrated plasma 104. In various embodiments, the water concentration is maintained in the range of up to approximately 0.5% w/w. According to various embodiments, freeze-drying may be employed to dehydrate the whole plasma.

Next, a predetermined quantity of the dehydrated plasma may be stored in plasma container 102. In various embodiments, dehydrated plasma 102 may be sterilized. In various embodiments, plasma container 102 may be labeled with tag 106.

Next, following an injury to the donor, at least a portion of the container may be filled with a predetermined volume of liquid to rehydrate the dehydrated plasma 104. In various embodiments of the present invention, the liquid may comprise sterile water for injection. In further embodiments, the predetermined volume of liquid may be in the range of 400 ml to 2000.

In an aspect of the invention, the rehydrated whole plasma may be filtered to remove particulates (i.e., using filter 112). In another aspect of the invention, the donor may be identified as the source of the dehydrated plasma using, for example, the donor identifying information included with tag 108. Following such identification, at least a portion of the rehydrated whole plasma may be administered to the donor.

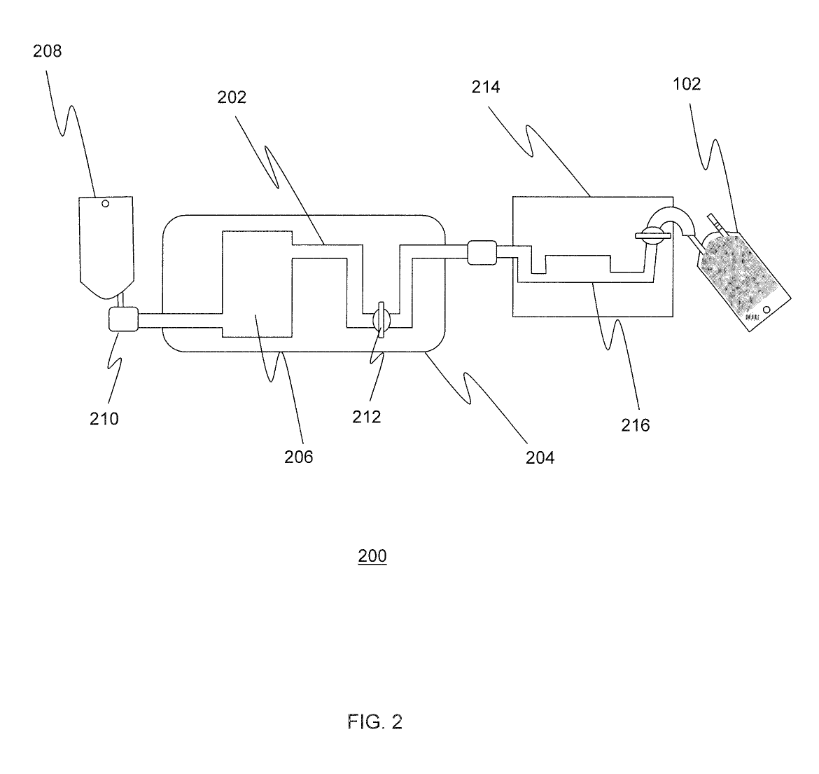

Referring to FIG. 2, wherein a simplified drawing illustrating a system for separating whole plasma from a sample of blood, in accordance with embodiments of the present invention, is shown. In accordance with various embodiments, system 200 may include a functionally closed processing article 202 and an automated separator apparatus 204 to monitor and automate the procedure. For such embodiments, processing article 202 may define a flow path through separator apparatus 204. Processing article 202 may be sterile and/or disposable. In various embodiments, processing article 202 may be based on a centrifugal processing chamber 206 whose volume can be varied during operation, allowing a user to adjust the exact quantity of blood to process. In various embodiments, the centrifugal processing chamber 206 may be connected to a set of bags and tubing lines for the collection of the separated components. A blood bag 208 containing the blood to process may be connected processing article 202 through the use of a sterile connecting device 210, or an aseptic connection under laminar flow. In other embodiments, it may be possible to have blood bag 208 prefilled with anticoagulant and/or preconnected to processing article 202.

A tubing line selection for directing separated blood products into proper collection bags may accomplished by one or more stopcock(s) 212 that can be arranged in a manifold array, or by a single multiport rotational valve. Such an arrangement may minimize or eliminate cross-contamination between adjacent lines in contrast to using standard pinch valves.

In accordance with various embodiments of the present invention, separator apparatus 204 may cooperate with instrumentation for monitoring and automating the process.

Separation system 200 may offer significant advantages over manual processing techniques. Processing article 202 may be a functionally closed system, avoiding any risk of contaminating the product during manipulation. The process steps may be fully automated, through a microprocessor based control system, with ability to vary the main parameters, like centrifugation speed, centrifugation time, speed of introduction and extraction, volume to collect, etc. The instrumentation may be very compact and portable, ideal for the decentralized processing and individualized processing of autologous plasma.

In various embodiments, separation system 200 may include a freeze-drying apparatus 214. In such embodiments, automated separator apparatus 204 may be coupled to freeze-drying apparatus 214. In various embodiments, freeze-drying apparatus 214 may include a freeze-dry kit 216. Freeze-drying apparatus 214 and/or freeze-dry kit 216 may be coupled to separator apparatus 204 and/or processing article 202 through the use of one or more sterile connecting device. Freeze-dry kit 216 may be sterile and/or disposable. In various embodiments, freeze-dry kit 216 may define a flow path through freeze-drying apparatus 214. In various embodiments, freeze-dry kit 216 may be based on an enclosed, single-use, autoclavable, disposable freeze-dry container, such as a GORE™ LYOGUARD® Freeze-Drying Tray.

In further embodiments, freeze-dry kit 216 may be connectable to plasma container 102 through the use of one or more sterile connecting device. Thus, dehydrated plasma 104 may be dispensed into plasma container 102 uncontaminated.

In accordance with various embodiments of the present invention, freeze-drying apparatus 214 may cooperate with instrumentation for monitoring and automating the freeze-dry process.

### Example

Preparation of the Lyophilized Plasma. A total of 5 L of whole porcine plasma was filtered through 2 layers of kitchen grade cheese cloth in ˜400 mL aliquots to remove excess fat from the plasma, yielding 3850 mL of usable plasma. The plasma was put into GORE™ LYOGUARD® trays and place into the freeze drier on a 4-day cycle starting at −40° C. After four days the freeze dried plasma formed a brittle cake. The cake was manual broken apart and ground to a fine powder.

Preparation of the Empty IV Bag. An empty 250 mL IV bag was obtained. The bag has two ports extending from the base of the bag: 1) Outlet port (5 cm long and 1 cm in diameter) for IV line insertion sealed with a breakable plastic membrane and covered with a sterile blue cap 2) Inlet port (3.5 cm long and 1 cm in diameter) for medication injection sealed with a breakable plastic membrane about half-way up the length of the port, and capped at the end with a rubber stopper which is sealed with a plastic tension ring. The rubber stopper and plastic ring was carefully removed from the Inlet port. The membrane in the port chamber was broken using an IV line, to allow for an opening into the bag.

Filling the Bag. A small diameter funnel was attached to the inlet port end of the bag, allowing for a tight seal around the funnel. The lyophilized plasma was divided up into powder aliquots such that when reconstituted with 250 mL of solvent it would be the same concentrations as the starting plasma. The powder aliquots were slowly funneled into the empty bags using a rod to break up any large powder cakes that would clog the funnel.

What is claimed is:

1. A kit for storing a therapeutic dose of autologous plasma obtained from a single donor prior to injury, comprising:
a. a first container filled with a predetermined quantity of dehydrated plasma obtained from the single donor; and
b. a tag having identifying information to identify the donor as the source of the dehydrated plasma.

2. The kit of claim 1, wherein the first container comprises a fluid inlet for filling the container with fluid to rehydrate the autologous dehydrated plasma and a fluid outlet for administering rehydrated autologous plasma to the donor.

3. The kit of claim 2, wherein the fluid outlet comprises a filter for removing dehydrated plasma particulates.

4. The kit of claim 1, further comprising a second container for storing the first container.

5. The kit of claim 1, wherein the dehydrated plasma is sterile

6. The kit of claim 1, wherein the volume of the first container is in the range of 400 ml to 2000 ml.

7. The kit of claim 1, wherein the predetermined quantity of dehydrated plasma is in the range of 30 g to 140 g.

8. A method for administering a therapeutic dose of autologous plasma to a single donor following injury, comprising:
a. providing a kit according to any one of claims 1-7;
b. rehydrating the plasma powder with a liquid; and
c. administering the rehydrated plasma to the injured donor.

9. The method of claim 8, further comprising identifying the donor as the source of the dehydrated plasma prior to said administering the rehydrated autologous plasma to the injured donor.

10. The method according to claim 8, wherein a predetermined volume of liquid is added to the plasma powder.

11. The method of claim 10, wherein the predetermined volume of liquid is in the range of 400 ml to 2000 ml.

12. The method of claim 8, wherein the liquid is sterile water for injection.

13. A method for preparing and storing an autologous plasma sample, comprising:
a. obtaining a sample of blood from a single donor;
b. separating whole plasma from the sample of blood;
c. dehydrating the whole plasma to form a dehydrated plasma;
d. storing a predetermined quantity of the dehydrated plasma in a container; and
e. labeling the container with a tracking label having identifying information to match the plasma to the single donor.

14. The method of claim 13, wherein the predetermined quantity of the powder is in the container is in the range of 30 g to 140 g.

15. The method of claim 13, further comprising sterilizing the dehydrated plasma.

16. The method of claim 13, wherein said dehydrating the whole plasma comprises freeze-drying the whole plasma.

17. The method of claim 13, wherein the volume of the container is in the range of 400 ml to 2000 ml.

18. The method of claim 13, wherein the container comprises a fluid inlet for filling the container with fluid to rehydrate the autologous dehydrated plasma and a fluid outlet for administering rehydrated autologous plasma to the injured donor

19. The method of claim 18, wherein the fluid outlet comprises a filter for removing unhydrated dehydrated plasma particulates.
